(19) United States
(12) Patent Application Publication
(10) Pub. No.: US 2010/0319693 A1
(43) Pub. Date: Dec. 23, 2010

(54) FLUID PRODUCT DISPENSING DEVICE

(75) Inventors: Christophe Fagot, Mezy (FR); Xavier Donnette, Plaisir (FR); Jean-Marc Pardonge, Les Authieux Sur Port Saint Ouen (FR)

Correspondence Address: SUGHRUE MION, PLLC, 2100 PENNSYLVANIA AVENUE, N.W., SUITE 800, WASHINGTON, DC, 20037, US

(73) Assignee: VALOIS SAS, Le Neubourg (FR)

(21) Appl. No.: 12/517,967

(22) PCT Filed: Dec. 10, 2007
(86) PCT No.: PCT/FR2007/052463

§ 371 Date: Jun. 5, 2009

(30) Foreign Application Priority Data
Dec. 11, 2006 (FR) 0655411

(51) Int. Cl. A61M 15/00

(52) U.S. Cl. 128/203.15; 128/203.21

(57) ABSTRACT

A fluid dispenser device including a body (10) that is provided with a dispenser opening (1) and at least one cover element (11; 12) that is displaceable between a closed position, in which it covers said dispenser orifice (1), and an open position, in which it uncovers said dispenser orifice (1), said device further including: an elongate flexible strip (20) supporting a plurality of reservoirs (21) each containing a dose of fluid or powder; reservoir-opening means (30) for opening a respective reservoir on each actuation; and displacement means (40) for displacing said flexible strip (20) before and/or during and/or after each actuation, so as to bring a full reservoir into register with said reservoir-opening means, the leading end (1) of said flexible strip (25), in the displacement direction of said strip, being fastened to a receiver element (20) that is rotatably mounted relative to said body (50), said receiver element (50) being adapted to exert a traction force on said elongate strip (20).

The present invention relates to a fluid dispenser device, and more particularly to a dry-powder inhaler.

Inhalers are well known in the prior art. Various types exist. A first type of inhaler contains a reservoir receiving many doses of powder, the inhaler being provided with metering means making it possible, on each actuation, to remove one dose of said powder from the reservoir, so as to bring said dose into an expulsion duct in order to be dispensed to the user. Inhalers including individual reservoirs, such as capsules, that are loaded into the inhaler just before said inhaler is used are also described in the prior art. The advantage of such devices is that it is not necessary to store all of the doses inside the appliance, such that said appliance can be compact. However, the inhaler is more difficult to use, since the user is obliged to load a capsule into the inhaler before each use. Another type of inhaler consists in packaging the doses of powder in individual predosed reservoirs, then in opening one of the reservoirs each time the inhaler is actuated. That implementation seals the powder more effectively since each dose is opened only when it is about to be expelled. In order to make such individual reservoirs, various techniques have already been proposed, such as an elongate blister strip or blisters disposed on a rotary circular disk. All existing types of inhalers, including those described above, present both advantages and drawbacks associated with their structures and with their types of operation. Thus, with certain inhalers, there is the problem of accuracy and of reproducibility for the dose on each actuation. In addition, the effectiveness of the dispensing, i.e. the fraction of the dose that effectively penetrates into the user's lungs in order to have a beneficial therapeutic effect, is also a problem that exists with a certain number of inhalers. A solution for solving that specific problem has been to synchronize the expulsion of the dose with the inhalation of the patient. Once again, that can create drawbacks, in particular in that type of device, the dose is generally loaded into an expulsion duct before inhalation, then expulsion is synchronized with inhalation. That means that if the user drops, shakes, or manipulates the inhaler in an undesirable or inappropriate manner between the moment when the user loads the dose (either from a multidose reservoir or from an individual reservoir) and the moment when the user inhales, then the user risks losing all or part of the dose, with said dose possibly being spread about inside the appliance. In that event, there can exist a high risk of overdosing the next time the device is used. The user who realizes that the dose is not complete will load a new dose into the appliance, and while the new dose is being inhaled, a fraction of the previous dose that was lost in the appliance could thus be expelled at the same time as the new dose, thereby causing an overdose. In the treatments envisaged, such overdosing can be very harmful, and the authorities in all countries are issuing ever-stricter requirements to limit the risk of overdosing as much as possible. With regard to opening the individual reservoirs, it has been proposed to peel off or to unstick the closure layer. That presents the drawback of difficulty in controlling the forces to be applied in order to guarantee complete opening, without running the risk of opening the next reservoir, particularly if the opening means need to be actuated by inhalation. Another problem that occurs with inhalers provided with blister strips is connected to the displacement of the strip, and to the storage of the used portion of the strip. Thus, depending on the length of the strip, a large amount of space can turn out to be necessary, and any blockage of the blister strip can prevent the inhaler from operating properly.

An object of the present invention is to provide a fluid dispenser device, in particular a dry-powder inhaler, that does not have the above-mentioned drawbacks.

In particular, an object of the present invention is to provide such an inhaler that is simple and inexpensive to manufacture and to assemble, that is reliable in use, guaranteeing metering accuracy and metering reproducibility on each actuation, providing an optimum yield with regard to the effectiveness of the treatment, by making it possible to dispense a substantial fraction of the dose to the zones to be treated, in particular the lungs, avoiding, in safe and effective manner, any risk of overdosing, and that is as compact as possible, while guaranteeing sealing and absolute integrity of all of the doses up to their expulsion.

Another object of the present invention is to provide such an inhaler provided with a blister strip, in which the storage of the used strip portion is optimized, and the risk of the strip blocking is minimized.

The present invention thus provides a fluid dispenser device including a body that is provided with a dispenser opening and at least one cover element that is displaceable between a closed position, in which it covers said dispenser orifice, and an open position, in which it uncovers said dispenser orifice, said device further including: an elongate flexible strip supporting a plurality of reservoirs each containing a dose of fluid or powder; reservoir-opening means for opening a respective reservoir on each actuation; and displacement means for displacing said flexible strip before and/or during and/or after each actuation, so as to bring a full reservoir into register with said reservoir-opening means, the leading end of said flexible strip, in the displacement direction of said strip, being fastened to a receiver element that is rotatably mounted relative to said body, said receiver element being adapted to exert a traction force on said elongate strip.

Advantageously, said rotary receiver element is caused to turn while said at least one cover element is being opened and/or closed.

Advantageously, said rotary receiver element includes a set of teeth that co-operates with an actuator member that is secured to at least one cover element.

Advantageously, said set of teeth co-operates with an anti-return device, such as a finger, so as to prevent said rotary receiver element from turning in the direction opposite to that which is imparted thereto by said actuator member.

Advantageously, said actuator member comprises a toothed rod which, in one of its directions of displacement, causes said rotary receiver element to turn, and, in its other direction of displacement, slides over said set of teeth without causing the receiver element to turn.

Advantageously, said rotary receiver element is caused to turn while said at least one cover element is being opened.

Advantageously, said rotary receiver element is caused to turn while said at least one cover element is being closed.

Advantageously, said rotary receiver element co-operates with a primed spring, in particular a helical spring, that continuously urges it to turn, such that said receiver element continuously exerts traction on the leading end of the elongate strip.

Advantageously, said spring is preprimed while the device is being assembled, such that it exerts a maximum force on said rotary receiver element when first used, said force decreasing progressively each time the device is actuated.

Advantageously, said opening means comprise a needle that does not move relative to said body, a reservoir being displaced against said needle each time the device is actuated, said needle penetrating into said reservoir so as to empty it by means of an inhalation flow.

Advantageously, said opening means are controlled by the user inhaling, such that the reservoir is opened and emptied simultaneously by said inhalation flow.

These characteristics and advantages and others of the present invention appear more clearly from the following detailed description, given by way of non-limiting example, and with reference to the accompanying drawings, in which:

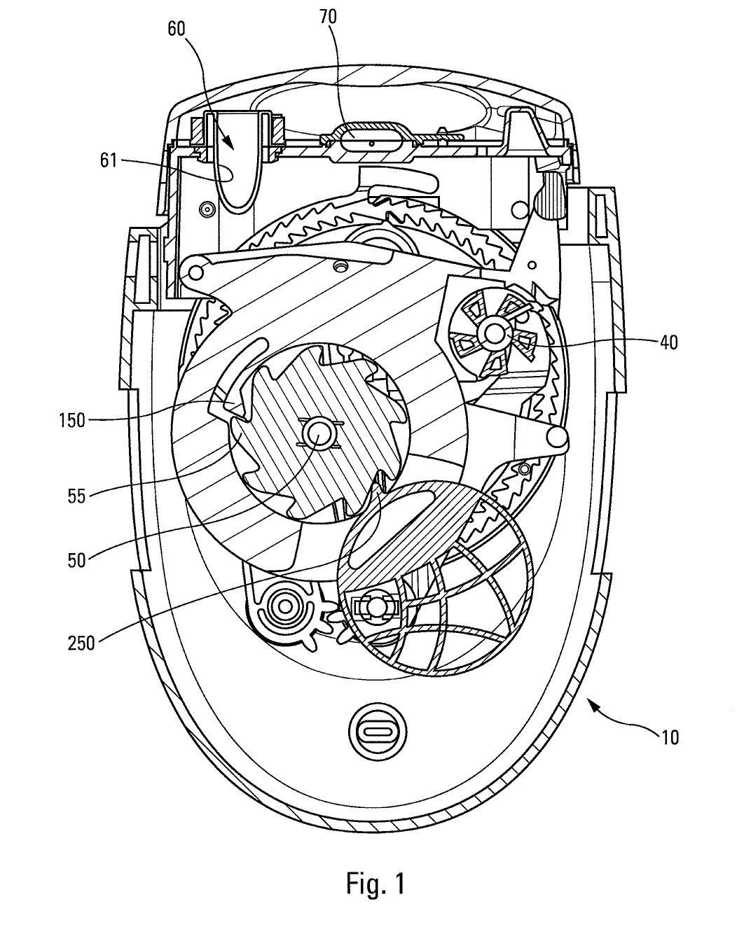

FIG. 1 is a diagrammatic section view showing a dispenser device constituting a first advantageous embodiment of the invention;

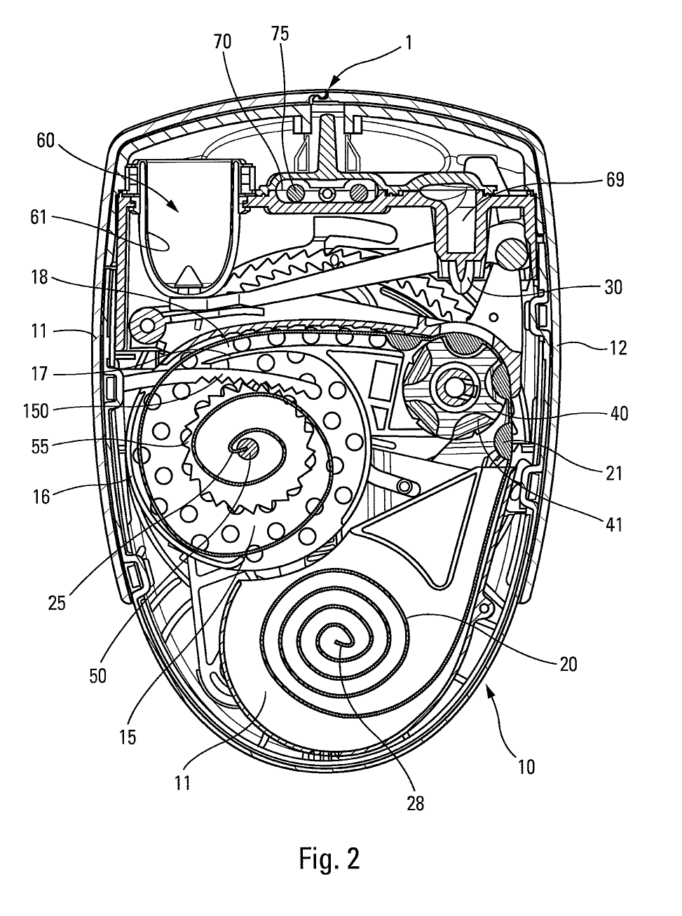

FIG. 2 is a view similar to the view in FIG. 1, showing a variant embodiment of the invention, in the closed position;

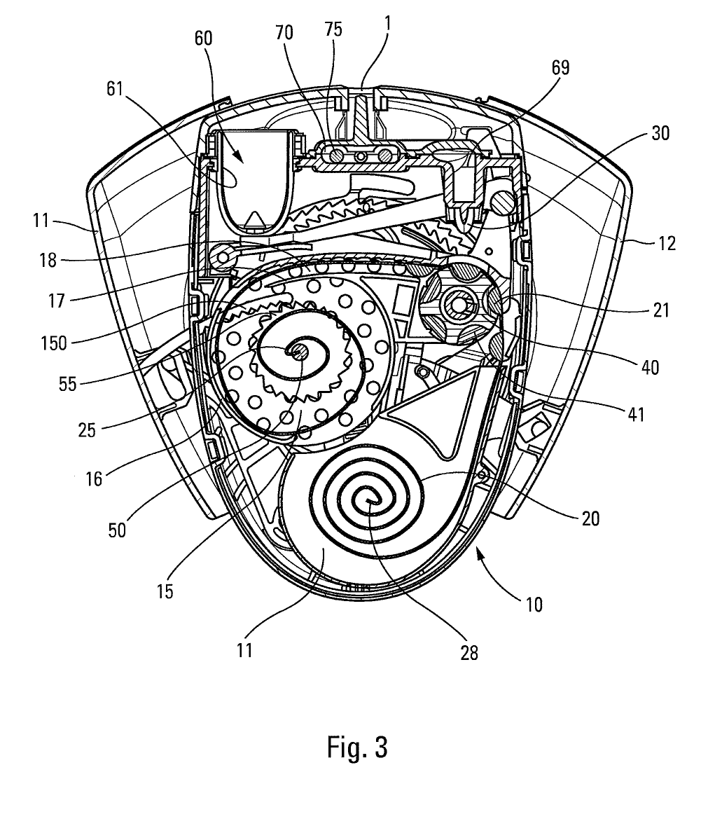

FIG. 3 is a view similar to the view in FIG. 2, in the open position; and

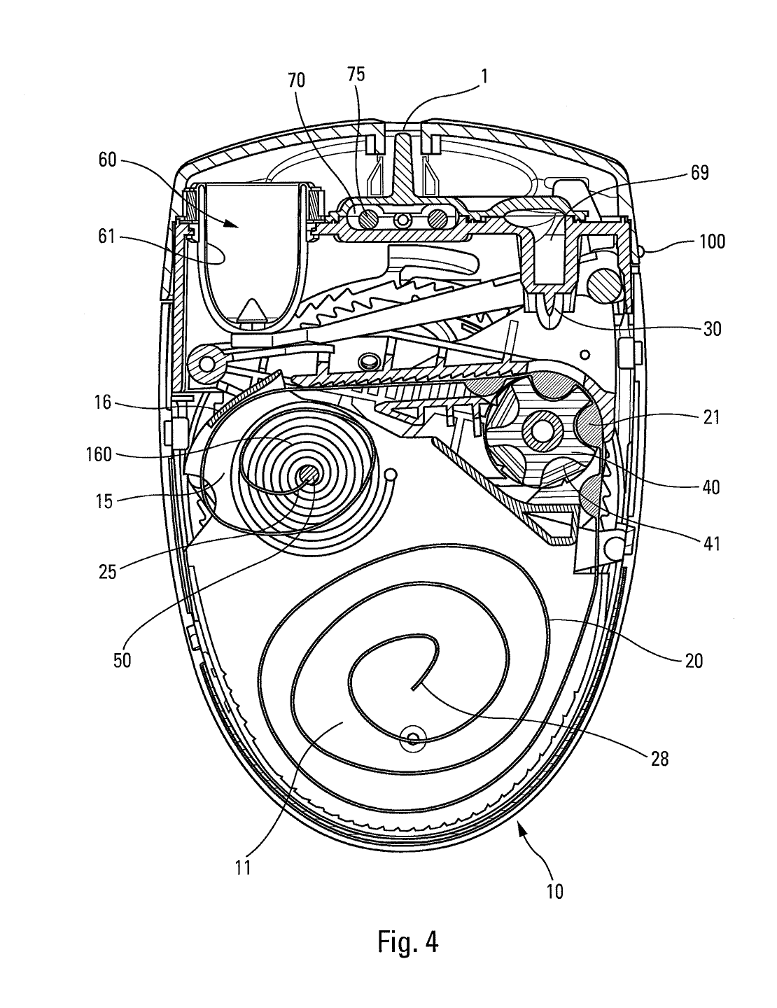

FIG. 4 is a view similar to the views in FIGS. 1 to 3, showing another advantageous embodiment of the invention.

FIGS. 1 to 3 show two advantageous variant embodiments of a dry-powder inhaler. The inhaler includes a body 10 on which there can be slidably mounted two cover-forming portions 11, 12 that are adapted to be opened so as to open and prime the device. The body 10 can be of approximately rounded shape as shown in the figures, but it could have any other appropriate shape. The body 10 includes a mouthpiece or inhaler endpiece 1 that defines a dispenser orifice through which the user inhales while the device is being actuated. The covers 11, 12 can open by pivoting about a common pivot axis, but any other means of opening the device can be envisaged. In a variant, the device could include a single cover instead of two.

Inside the body 10, there is provided a strip 20 of individual reservoirs 21, also known as blisters, that is made in the form of an elongate strip 20 on which the blisters 21 are disposed one behind the other in known manner. The blisters 21 are not shown along the entire strip 20, so as to simplify the drawings for the purpose of clarity. The blister strip 20 is advantageously constituted by a base layer or wall that forms the cavities 21 that receive the doses of powder, and by a closure layer or wall that closes each of said blisters 21 in leaktight manner. Before first use, the blister strip 20 can be rolled-up inside the body 10, preferably in a storage housing 11, and strip drive means 40 are provided for progressively unrolling the blister strip and for bringing an individual reservoir or respective blister 21 into a dispensing position each time the device is actuated. The strip portion 25 including the empty reservoirs is advantageously adapted to be rolled up in another location of said body 10, preferably a reception housing 15, as described in greater detail below.

The inhaler includes reservoir-opening means 30 preferably comprising perforator and/or cutter means for perforating and/or cutting the closure layer of the blisters. For example, the reservoir-opening means advantageously comprise a needle 30 that preferably does not move relative to the body 10, and against which a respective blister 21 is displaced on each actuation. The blister is thus perforated by said needle that penetrates into said blister so as to expel the powder by means of the user inhaling.

Displacement means 40 are also provided in the device and are adapted to displace the blister strip 20 before and/or during and/or after each actuation of the device. Advantageously, the displacement means 40 are also adapted to displace the reservoir 21 to be emptied against said perforator and/or cutter means 30 during actuation. The displacement means 40 can be urged by a resilient element, such as a spring or any other equivalent resilient element, said resilient element possibly being preprimed while the device is being opened. The displacement means 40 preferably comprise an indexer wheel 40 that receives and guides the blisters. Turning the wheel 40 causes the blister strip 20 to advance. In a particular angular position, a given reservoir 21 is always in a position to be opened by the opening means 30. Advantageously, rotary positioning means of said indexer wheel 40 can be provided for precisely determining its angular position after each turn. In an advantageous variant, the positioning means can comprise a projection or finger having an end that co-operates elastically with notches provided around said wheel 40. A complementary wheel could possibly be provided so as to held to guide and/or to drive the blister strip 20.

An actuation cycle of the device could be as follows. During opening of the device, the two cover-forming lateral portions 11, 12 are moved away from each other by pivoting about the body so as to open the device and thus prime the device. In this position, the indexer wheel 40 cannot be displaced towards the needle 30 since it is retained by appropriate blocking means 100. It is while the user is inhaling through the mouthpiece 1 that the blocking means are unblocked, thereby causing said indexer wheel 40 to be displaced towards the needle 30, and thus causing a reservoir 21 to be opened.

In the embodiments shown, the reservoir 21 is displaced towards its open position so as to be opened by the needle 30 that does not move relative to the body. However, it can be envisaged that the needle can also be movable during the stage of opening the reservoir 21. For example, the needle could be displaced towards the reservoir 21 while the reservoir 21 is displaced towards the needle. In another variant, it is also possible to envisage that the reservoir 21 and the needle are displaced in the same direction during actuation, the reservoir 21 being displaced quicker in said direction, such that it comes into contact with the needle so as to be opened.

As explained above, it is desirable for the opening means to be actuated by the user inhaling. In order to trigger the reservoir opening means by inhalation, an inhalation trigger system can be provided that advantageously comprises a unit 60 that is displaceable and/or deformable under the effect of inhalation, the unit 60 being adapted to release the blocking means 100. The unit 60 advantageously comprises a deformable air chamber 61. The inhalation by the user causes said deformable air chamber 61 to deform, thereby enabling said blocking means 100 to be released, and thus enabling the indexer wheel 40 and a respective reservoir 21 to be displaced towards its opening position. The reservoir 21 is thus opened only at the moment of inhalation, so that it is emptied simultaneously. Thus, there is no risk of any of the dose being lost between opening the reservoir and emptying it.

In a variant, other inhalation trigger means could also be used, e.g. using a pivotable valve flap that, while the user is inhaling, pivots under the effect of the suction created by the inhalation, with pivoting of the valve flap causing the blocking means blocking the movable support means to be released, thereby causing the reservoir to be displaced towards the opening means.

The inhaler further includes a dispenser chamber 70 for receiving the dose of powder after a respective reservoir 21 has been opened. The dispenser chamber 70 is advantageously provided with at least one bead 75 that is displaced inside said chamber 70 during inhalation so as to improve dispensing of the air and powder mixture after a reservoir 21 has been opened, in order to increase the effectiveness of the device.

It can be advantageous for the opening means 30, in particular the needle, to be formed directly on said dispenser chamber 70, e.g. at the end of a channel 69 leading to said chamber 70.

After inhalation, when the user closes the device, all of the components return to their initial rest position. The device is thus ready for a new cycle of use.

In an advantageous aspect of the inhaler, the individual reservoirs or blisters 21 are formed on an elongate strip 20 that, initially, is mainly stored in the form of a roll in a storage housing 11 inside the body 10 of the device. Advantageously, the rolled-up blister strip 20 is held by internal walls of said storage housing 11 without its trailing end 28 (in the direction of displacement of the blister strip 20) being fastened relative to said body 10, thereby making it easier to assemble the blister-strip roll inside the device. The blister strip 20 is displaced by the user, advantageously by means of the indexer wheel 40 that advantageously presents at least one, and preferably a plurality of recesses 41, having a shape that corresponds to the shape of the blisters. Thus, when the indexer wheel 40 turns, it advances the blister strip 20. Naturally, in a variant or in additional manner, it is possible to use other means for advancing the blister strip, e.g. providing a profile on the longitudinal lateral edges of the blister strip, said profile being adapted to co-operate with appropriate drive means. In addition, holes formed along the lateral edges of the blister strip could also be used to cause the blister strip to advance by means of toothed wheels co-operating with said holes.

After opening one or more blisters, the blister-strip portion with the empty reservoirs must be suitable for being stored in easy and compact manner in the device, avoiding any risk of blocking. Advantageously, the used blister strip is rolled-up automatically, once again forming a roll. In the invention, the leading end 25 of the blister strip 20 is fastened to a receiver element 50 that is rotatably mounted relative to the body 10.

To ensure that the leading portion of the blister strip 20, namely the portion including the empty blisters, is rolled up properly in the reception housing 15, the rotary receiver element 50 is adapted to exert a traction force on the strip 20, in particular on its leading end 25. Thus, any risk is avoided of the strip being rolled up poorly, e.g. folding up concertina-like, etc.

FIGS. 1 to 3 show two variant embodiments, in which the traction force is exerted either while the covers 11, 12 are being opened, or while they are being closed. In the two variants, an actuator member 150 is adapted to co-operate with a set of teeth 55 of the receiver element 50, said actuator member 150 being secured to a cover element. Thus, while said covers are being moved, said actuator member 150 co-operates with the set of teeth so as to turn the receiver element 50.

Preferably, a non-return system 250, such as a finger as shown in FIG. 1, also co-operates with the receiver element 50, in particular with the set of teeth 55, so as to prevent the receiver element 50 from turning in the opposite direction to the direction imparted by the actuator member 150. For example, if the actuator member 150 turns the receiver element 50 while the covers are being opened, then the non-return finger 250 blocks the receiver element 50 while the covers are being closed, and vice versa. In FIG. 1, the actuator member 150 is a flexible finger that, in one direction, co-operates with the set of teeth 55 so as to turn the receiver element 50, and in the other direction slides over the set of teeth. In FIGS. 2 and 3, the actuator member 150 is a toothed rod that operates in similar manner. It should be observed that the non-return system is not visible in FIGS. 2 and 3, but preferably it is also provided.

FIG. 4 shows another embodiment of the invention in which the traction of the receiver element 50 on the strip 20 is not linked to the manipulation of the covers. In this variant, a primed spring 160, preferably a helical spring, is connected to said receiver element 50 so as to urge it continuously to turn. In this way, the traction exerted on the strip 20 is continuous, guaranteeing that the strip is always under tension and properly rolled up. With a helical spring, said helical spring can be preprimed during assembly, i.e. rolled up. In this way, the traction is at its maximum at the start of use, and progressively decreases on each actuation as a result of the spring progressively relaxing.

Advantageously, the receiver element 50 is disposed approximately at the center of the reception housing 15. The reception housing can include guide walls, in particular an external guide wall 16 that is curved, e.g. cylindrical, and against which the blister strip 20 slides. An internal guide wall 17 can also be provided at the inlet to the reception housing 15, and preferably extends approximately parallel to the external guide wall 16, so as to form a guide channel 18 for the blister strip 20. The guide walls 16, 17 further facilitate proper rolling up of the blister strip 20 around the receiver element 50.

The present invention therefore makes it possible to provide a dry-powder inhaler that performs the following functions in particular:
a plurality of individual doses of powder are stored in individual sealed reservoirs, e.g. 30 or 60 doses stored on a rolled-up strip;
the powder is released by perforation that is achieved by the user inhaling, the blister being perforated by means of an inhalation detector system that is coupled to a preprimed release system;
appropriately-shaped drive means are engaged with blisters so as to displace the blister strip on each actuation, and bring a new reservoir into a position in which it is to be opened by appropriate opening means; and
safe and reliable storage of the used portion of the strip, by being rolled up around a rotary element that is adapted to pull on the strip on each actuation.

Other functions are also provided by the device of the invention as described above. It should be observed that the various functions, even if they are shown as being provided simultaneously on the various embodiments of the inhaler, could be implemented separately. En particular, the inhalation trigger mechanism could be used independently of the type of reservoir-opening means, independently of the use of a dose indicator, independently of the way in which the individual reservoirs are arranged relative to one another, etc. The prepriming means and the inhalation trigger system could be made in some other way. The same applies for other component parts of the device.

Various modifications can also be envisaged by a person skilled in the art, without going beyond the ambit of the present invention, as defined by the accompanying claims.

1.-11. (canceled)

12. A fluid dispenser device including a body (10) that is provided with a dispenser opening (1) and at least one cover element (11; 12) that is displaceable between a closed position, in which it covers said dispenser orifice (1), and an open position, in which it uncovers said dispenser orifice (1), said device further including: an elongate flexible strip (20) supporting a plurality of reservoirs (21) each containing a dose of fluid or powder; reservoir-opening means (30) for opening a respective reservoir on each actuation; and displacement means (40) for displacing said flexible strip (20) before and/or during and/or after each actuation, so as to bring a full reservoir into register with said reservoir-opening means, the leading end (25) of said flexible strip (20), in the displacement direction of said strip, being fastened to a receiver element (50) that is rotatably mounted relative to said body (10), said receiver element (50) being adapted to exert a traction force on said elongate strip (20), said rotary receiver element (50) being caused to turn while said at least one cover element (11, 12) is being opened and/or closed, said device being characterized in that said rotary receiver element (50) includes a set of teeth (55) that co-operates with an actuator member (150) that is secured to at least one cover element (11, 12), said actuator member (150) comprising a toothed rod (160) which, in one of its directions of displacement, causes said rotary receiver element (50) to turn, and, in its other direction of displacement, slides over said set of teeth (55) without causing the receiver element (150) to turn.

13. A device according to claim 12, in which said set of teeth (55) co-operates with an anti-return device (250), such as a finger, so as to prevent said rotary receiver element (50) from turning in the direction opposite to that which is imparted thereto by said actuator member (150).

14. A device according to claim 12, in which said rotary receiver element (50) is caused to turn while said at least one cover element (11, 12) is being opened.

15. A device according to claim 12, in which said rotary receiver element (50) is caused to turn while said at least one cover element (11, 12) is being closed.

16. A device according to claim 12, in which said opening means (30) comprise a needle (30) that does not move relative to said body (10), a reservoir (21) being displaced against said needle each time the device is actuated, said needle penetrating into said reservoir (21) so as to empty it by means of an inhalation flow.

17. A device according to claim 12, in which said opening means (30) are controlled by the user inhaling, such that the reservoir (21) is opened and emptied simultaneously by said inhalation flow.
